(19) United States
(12) Patent Application Publication
(10) Pub. No.: US 2013/0122119 A1
(43) Pub. Date: May 16, 2013

(54) PHYLLANTHUS AMARUS COMPOSITIONS AND METHOD OF EXTRACTING SAME

(71) Applicant: Natreon, Inc., New Brunswick, NJ (US)

(72) Inventors: Shibnath Ghosal, Kolkata (IN); Muruganandam Veeraragavan, Kolkata (IN); Sanyasi R. Kalidindi, East Brunswick, NJ (US)

(73) Assignee: NATREON, INC., New Brunswick, NJ (US)

(21) Appl. No.: 13/666,637

(22) Filed: Nov. 1, 2012

Related U.S. Application Data
(60) Provisional application No. 61/554,235, filed on Nov. 1, 2011.

(51) Int. Cl. A61K 36/185

(52) U.S. Cl. CPC A61K 36/185
USPC 424/725; 264/5

(57) ABSTRACT

An enriched hydrolyzable tannin blend derived from Phyllanthus amarus is provided. An optimized aqueous extraction method for Phyllanthus amarus is provided to maximize the levels of bioactive hydrolyzable tannins including corilagin, geraniin, nirurin and other low molecular weight hydrolyzable tannoids. The method produces a Phyllanthus amarus extract containing a hydrolyzable tannin blend as an amorphous dry powder. In an embodiment, the Phyllanthus amarus extract contains 7-13% by weight corilagin, 3.5-10% nirurin, 1-2% geraniin, and 9-20% low molecular weight hydrolyzable tannoids. In another embodiment, the Phyllanthus amarus extract contains 12-20% geraniin and 12-20% low molecular weight hydrolyzable tannoids. Potential uses of said enriched hydrolyzable tannin compositions for hepatoprotection in a human subject are described herein.

This application claims the benefit of earlier filed U.S. Provisional Application No. 61/554,235, filed on Nov. 1, 2011, which is hereby incorporated by reference herein.

### FIELD OF THE INVENTION

The present invention relates to an enriched hydrolyzable tannin blend derived from Phyllanthus amarus. The invention further relates to a method for extracting Phyllanthus amarus to obtain a hydrolyzable tannin powder enriched with corilagin and geraniin. This invention further relates to use of said enriched hydrolyzable tannin compositions for hepatoprotection in a human subject.

### BACKGROUND

Phyllanthus amarus (P. amarus), also known as Phyllanthus niruri, is a widespread tropical plant commonly found in the hotter coastal regions of India. It belongs to the Family Euphorbiaceae.

Traditionally, all parts of the plant can be used medicinally, including leaves, tender aerial parts, and roots. The plant has been known for centuries for treatment of jaundice, and is a commonly used as a household remedy in India. See, M. S. Premila, Ayurvedic Herbs: A Clinical Guide to the Healing Plants of Traditional Indian Medicine (New York: The Haworth Press, 2007).

P. amarus has been a very important part of traditional medicine in many countries of the world. It is reported to have anti-oxidant, anti-inflammatory, anti-cancer, hypoglycemic, and hepatoprotective properties.

P. amarus has been studied extensively, following the discovery that medicinal preparations thereof can bind the hepatitis B virus surface antigen (HBsAg) (S. P. Thyagarajan, et al., Indian J. Med. Res. (1982) 76(suppl.):124-130). The aerial parts of P. amarus bear official status in the Indian Herbal Pharmacopoeia, for antiviral activity (Vol. II, pp. 85-92; Mumbai: Indian Drug Manuf. Assoc. and Jammu Tawi: Regional Res. Lab., 1999).

P. amarus contains a rich blend of polyphenolics comprising lignans, tannins, flavonoids, and also sterols, and alkaloids. The lignans phyllanthin and hypophyllanthin have been shown to be hepatoprotective against carbon tetrachloride (CCl₄)-induced hepatotoxicity in primary cultured hepatocytes (K. V. Syamsundar, et al., J. Ethnopharmacol. (1985) 14:41-44).

As mentioned above, P. amarus has been shown to possess in vitro antiviral activity against hepatitis B virus (HBV). The plant extracts have been shown to inhibit HBs-Anti HBs reaction (i.e., HBV-HBsAg) and inhibit HBV DNA polymerase activity. In cell culture it downregulates HBV mRNA transcription and replication, and inhibits HBV enhancer I activity with respect to cellular transcription factors. (R. Mehrotra, et al., Indian J. Med. Res. (1991) 93:71-73; C-D Lee, et al., Eur. J. Clin. Invest. (1996) 26:1069-1076; M. Ott, et al., Eur. J. Clin. Invest. (1997) 27:908-915.) In a double-blind, placebo-controlled trial, 59% of chronic hepatitis B patients became HBsAg negative after ingesting 200 mg powder of aerial parts of P. amarus three times a day for 1 month, while seroconversion in the placebo group was 4% (S. P. Thyagarajan, et al., The Lancet (1988) (October 1) 2:764-766). And despite the fact that there have also been negative clinical trials, P. amarus must be considered a plant of potential use in the treatment of viral hepatitis B, among other diseases. However, further investigation is needed, especially with regard to choice of plant material, method of processing, dosages, and period of treatment.

The anti-oxidant potential of an aqueous extract of P. amarus has been studied in rats wherein a significant decrease in plasma lipid peroxidation and a significant increase in: plasma vitamin C, uric acid level, reduced glutathione (GSH) level, glutathione peroxidase activity, catalase and superoxide dismutase activities has been demonstrated. It was also shown that this extract was devoid of genotoxicity and had a significant protective effect against hydrogen peroxide, streptozocin and nitric oxide-induced lymphocyte DNA damage (R. Karuna, et al., Indian J. Pharmacol. (2009) April; 41(2):64-7).

The effects of one methanolic extract of P. amarus on different phases of inflammation were examined. Investigations were performed using different phlogistic agents-induced paw edema, carrageenan-induced air-pouch inflammation and cotton pellet granuloma in rats. Methanolic extract of P. amarus significantly inhibited carrageenan, bradykinin, serotonin and prostaglandin E1-induced paw edema, but failed to inhibit the histamine-induced paw edema. Maximum inhibition was observed in prostaglandin E1-induced paw edema. In the carrageenan air-pouch model, a methanol extract of P. amarus significantly reduced the volume of exudate and migration of neutrophils and monocytes. The extract significantly decreased formation of granuloma tissue in a chronic inflammation model. The study revealed that methanolic extracts of P. amarus inhibits all the phases of inflammation (M A Mahat, et al., Indian J Pharm Sci (2007) 69:33-36).

Oral administration of P. amarus was found to enhance the life span of leukemia-harboring animals and decrease the incidence of anemia. In this study, the authors also performed a series of hematological, biochemical, histopathological, and gene expression analyses to evaluate the effect of P. amarus administration on erythroleukemia initiation and progression. The data obtained indicate that P. amarus administration could significantly decrease the progression of erythroleukemia (K. B. Harikumar, et al., Integr Cancer Ther. (2009) September; 8(3):254-60. The same authors also reported the apoptotic effects of P. amarus against Dalton's lymphoma ascites (DLA) cells in cell cultures. P. amarus produced significant reduction in DLA cell viability. It also induced the formation of apoptotic bodies with characteristic features like plasma membrane invagination, elongation, fragmentation, and chromatin condensation. P. amarus at concentrations of 100 and 200 micrograms/mL is shown to induce DNA fragmentation. Gene expression analysis reveals that P. amarus induces the expression of caspase-3 and inhibits the expression of Bcl-2, which is an antiapoptotic protein, thus providing some insights into the possible mechanism by which P. amarus brings about apoptosis and growth inhibition in DLA cells (K. B. Harikumar, et al., Integr Cancer Ther. (2009) June; 8(2):190-4). Another study reported that hairy root extract of P. amarus induced apoptotic cell death in human breast cancer cells (P. GauriAbhyankara, et al., Innovative Food Science & Emerging Technologies, Volume 11, Issue 3, July 2010, Pages 526-532). The cytotoxic effect and the multidrug resistance reversing action of lignans from P. amarus was reported to suggest a potential action of P. amarus derivatives as multi-drug resistance reversing agents, mainly due to their ability to synergize with the action of conventional chemotherapeutics (D. F. Leite, et al., Planta Med. (2006) December; 72(15):1353-8).

Furthermore, P. amarus extract was found to show hepatoprotective effects by lowering the content of thiobarbituric acid reactive substances, enhancing the reduced glutathione level, and increasing the activities of antioxidant enzymes, glutathione peroxidase, glutathione-S-transferase, superoxide dismutase and catalase. Histopathological analyses of liver samples also confirmed the hepatoprotective value and antioxidant activity of the ethanolic extract of the herb, which was comparable to the standard antioxidant, ascorbic acid. The overall data indicated that P. amarus possesses a potent protective effect against aflatoxin B(1)-induced hepatic damage, and it was suggested that the main mechanism involved in the protection could be associated with its strong capability to reduce the intracellular level of reactive oxygen species by enhancing the level of both enzymatic and non-enzymatic antioxidants (F. Naaz, et al., J Ethnopharmacol. (2007) 113(3):503-9). A study was carried out on the hepatoprotective activity of P. amarus plant extract against carbon tetrachloride (CCl₄)-induced liver damage in female mice. Carbon tetrachloride administration caused a significant increase in liver and serum alanine transaminase (ALT), aspartate transaminase (AST), alkaline phosphatase (ALP) and acid phosphatase, while total protein content significantly decreased as compared to vehicle control. The effect was dose-dependent. Oral administration of aqueous extract of P. amarus along with carbon tetrachloride caused significant mitigation of CCl₄-induced changes (R. Krithika, et al., Acta Pol Pharm. (2009) July-August; 66(4):439-44). See also the results section below.

Carbon tetrachloride is one of the most commonly known hepatotoxins. It is also well documented that carbon tetrachloride is biotransformed under the action of cytochrome P₄₅₀ in the microsomal compartment of liver to trichloromethyl radical which readily reacts with molecular oxygen to form trichloromethylperoxy radical. Both of these radicals can bind covalently to the macromolecules and induce peroxidative degradation of the membrane lipids of endoplasmic reticulum which is rich in polyunsaturated fatty acids (Recknagael R., “Carbon tetrachloride hepatotoxicity,” Pharmacol. Review (1967) 19: 145-196). This leads to the formation of lipid peroxides followed by pathological changes such as depression of protein synthesis, elevated levels of serum marker enzymes such as serum glutamic-oxaloacetic transaminase (SGOT), serum glutamate pyruvate transaminase (SGPT) and ALP, depletion of glutathione content and catalase activity, and increase in lipid peroxidation. (O. Faroon, et al., “Carbon tetrachloride; Health effects toxicokinetics, human exposure and environmental fate,” Toxic Indust. Health. (1994) 10: 4 -20; H. J. Zimmerman and L. B. Seeff, “Enzymes in hepatic disease,” in: E. E. Goodly (Ed), Diagnostic Enzymology, pp. 24-26, (Lea and Febiger, Philadelphia, 1970); T. Kamiyama, et al., “Role of lipidperoxidation in acetaminophen induced hepatotoxicity; comparison with carbontetrachloride,” Toxicol Lett. (1993) 66: 7-12.) Although serum enzyme levels are not a direct measure of hepatic injury, they show the status of liver function. Elevated levels of serum enzymes are indicative of cellular leakage and loss of functional integrity of cell membrane in liver. Thus, lowering of the enzyme content in serum is a definitive indication of hepatoprotective action of a pharmaceutical or nutritional supplement composition.

High levels of SGOT indicates liver damage such as due to viral hepatitis. SGPT catalyses the conversion of alanine to pyruvate and glutamate and is released in a similar manner. Therefore, SGPT is more specific to the liver and a better parameter for detecting liver damage.

Additionally, the decomposition of lipid hydroperoxides leads to a wide variety of end products, one of which is malondialdehyde (MDA), which is now accepted as a reliable marker of lipid peroxidation (H. Ohkawa, et al., “Assay for lipid peroxides in animal tissues by thiobarbituric acid reaction,” Anal. Biochem. (1979) 95: 351-358).

Another study was undertaken to investigate the protective effect and possible mechanism of one aqueous extract from P. amarus (PA) on ethanol-induced hepatic injury in rats. In this in vitro study, PA (1-4 mg/ml) increased % MTT reduction assay (indicating increased cellular viability) and decreased the release of transaminases in rat primary cultured hepatocytes treated with ethanol. Hepatotoxic parameters studied in vivo included serum transaminases, serum triglyceride, hepatic triglyceride, tumor necrosis factor alpha (TNF-α), and interleukin-1 beta (IL-1β), together with histopathological examination. In one acute toxicity study, a single dose of PA (25, 50 and 75 mg/kg, p.o.) or SL (silymarin, a reference hepatoprotective agent, 5 mg/kg/day), 24 h before ethanol (5 g/kg/day, p.o.) lowered the ethanol-induced levels of transaminases. The 75 mg/kg PA dose gave the best result, similar to SL. Treatment of rats with PA (75 mg/kg/day, p.o.) or SL (5 mg/kg/day, p.o.) for 7 days, after 21 days of treatment with ethanol (4 g/kg/day, p.o.), enhanced liver cell recovery by bringing the levels of the transaminases, hepatic triglyceride, and TNF-α, back to normal. Histopathological observations confirmed the beneficial roles of PA and SL against ethanol-induced liver injury in rats (P. Pramyothin, et al., J. Ethnopharmacology (2007) 114(2):169-173). Possible mechanisms for these results may involve the antioxidant activity of PA and/or SL. In another study, a combination of silymarin and P. amarus showed synergistic effects for hepatoprotection; and, silymarin in combination with an ethanolic extract of P. amarus showed better activity due to the higher concentration of phyllanthin in an ethanolic extract in comparison to an aqueous extract of P. amarus (N. P. Yadav, et al., Phytomedicine (2008) 15(12):1053-61).

In view of the above, it would be desirable to provide a potent and therapeutically effective extract of P. amarus in a pharmaceutical or nutraceutical composition having improved properties for the treatment or prevention of diseases, in particular, liver and/or kidney diseases. It would also be desirable to provide an extract of P. amarus for use as a nutritional supplement.

If a way could be found to enhance or enrich the levels of bioactive tannins and/or tannoids including corilagin, geraniin, nirurin, and other low molecular weight hydrolyzable tannoids in a P. amarus extract, this would represent a valuable contribution to the art.

### SUMMARY OF THE INVENTION

An objective of the present invention is to develop an optimized extraction process to enrich the bioactive contents, including: corilagin, nirurin, and low molecular weight hydrolyzable tannoids (LMwHTs) in a P. amarus extract.

Another objective of the invention is to develop an optimized extraction process to enrich the bioactive contents, including: geraniin and LMwHTs in a P. amarus extract.

It is a further objective of the invention to develop an extraction process for P. amarus which is substantially aqueous. It is another objective of the invention to develop an extraction process for P. amarus which is essentially completely aqueous.

In one embodiment, a Phyllanthus amarus extract contains about 7-13% by weight corilagin, about 3.5-10% by weight nirurin, about 1-2% by weight geraniin, and about 9-20% by weight other low molecular weight hydrolyzable tannoids.

In another embodiment, the Phyllanthus amarus extract contains about 12-20% by weight geraniin, and about 12-20% by weight other low molecular weight hydrolyzable tannoids.

### BRIEF DESCRIPTION OF THE DRAWINGS

FIG. 1 depicts an exemplary chromatogram of P. amarus extract prepared in one embodiment as described using an HPLC analytical method (reversed phase C18, 0.1 phosphoric acid/acetonitrile eluant with UV detection at 270 nm) showing the standardized elution times for several principal bioactive components (corilagin, geraniin, nururin, and LMwHTs).

### DETAILED DESCRIPTION

In an embodiment, a Phyllanthus amarus extract containing a hydrolyzable tannin blend is provided. A method for extracting Phyllanthus amarus to obtain an enriched hydrolyzable tannin powder is also provided.

Studies cited above used whole extracts of P. amarus. However, P. amarus contains several bioactive components, including corilagin, geraniin, nirurin, lignans, and other low molecular weight hydrolyzable tannoids (LMwHTs). Many studies have also been done on the individual bioactives of P. amarus and are described below.

Tannins may be divided into two groups: (a) hydrolyzable tannoids (HTs), which are esters of a polyol or sugar, usually glucose, with one or more trihydroxybenzenecarboxylic acids (i.e., gallates), and (b) derivatives of procyanidins, flavanols or flavanones, so-called condensed tannins HTs are molecules with a polyol (generally D-glucose or its derivatives) as a central core. The hydroxyl groups of these carbohydrates may be partially or totally esterfied with phenolic carboxylic acids like gallic acid (gallotannins), ellagic acid (ellagitannins) or both (gallo-ellagitannins)

Corilagin, depicted in the compound of formula (1), is a tannoid (low Mw polyphenolic) member of the tannin family and has been found as a constituent in many medicinal plants. Corilagin is chemically named as beta-1-O-galloyl-3,6-(R)-hexahydroxydiphenoyl-D-glucose.

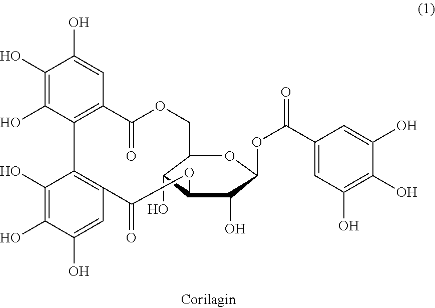 [SMILES: O=C(O[C@@H]1OC2COC(=O)c3cc(O)c(O)c(O)c3-c3c(cc(O)c(O)c3O)C(=O)O[C@@H](C1O)[C@@H]2O)c1cc(O)c(O)c(O)c1]

Corilagin has been studied extensively for its antibacterial, antiviral, antihypertensive, anti-inflammatory, antitumor, cardiovascular, and hepatoprotective activities. An extract of Arctostaphylos uva-ursi markedly reduced the MICs of β-lactam antibiotics, such as oxacillin and cefmetazole, against methicillin-resistant Staphylococcus aureus (M. Shimizu, et al., Antimicrob. Agents Chemother. (2001) (45) 11: 3198-3201). The authors isolated the effective compound in this particular extract and identified it as corilagin. Corilagin reduced the MICs of various βb -lactams by 100- to 2,000-fold but not the MICs of other antimicrobial agents tested. The effect of corilagin and oxacillin was observed to be synergistic. Corilagin showed a bactericidal action when added to the growth medium in combination with oxacillin. The antihypertensive effect of corilagin, being one of the ellagitannins purified from the seeds of Euphoria longana Lam. (Sapindaceae), was investigated in the spontaneously hypertensive rat (SHR). The results suggest that corilagin possesses the ability to lower blood pressure through the reduction of noradrenaline release and/or direct vasorelaxation. Anti-inflammatory effects of corilagin in herpes simplex virus 1 (HSV-1) encephalitis and HSV-1 infected microglias have been studied as well. It was concluded that corilagin has the potential to reduce HSV- 1-induced inflammatory insult to the brain, and its mode of action appears to be through the induction of apoptosis of microglias and reduction of cytokines production.

Corilagin has been found to have a protective effect on liver function and a restorative effect in cholestatic hepatitis by an anti-inflammatory pathway. The effects are mainly due to antagonizing proinflammatory cytokines and mediators, inhibiting oxidative damage, improving hepatic microcirculation, reducing impairment signals, and controlling neutrophil infiltration. Anti-inflammatory action of corilagin was further investigated. It was suggested that corilagin possesses potential anti-inflammatory activity not only by abating inflammatory impairment but also by promoting regression of inflammation (Lei Zhao, et al., International Immunopharmacology (2008) 8:1059-1064).

Corilagin (beta-1-O-galloyl-3,6-(R)-hexahydroxydiphenoyl-D-glucose), and its analogue Dgg16 (1,6-di-O-galloyl-beta-D-glucose) were shown to be effective in inhibiting the progress of atherosclerosis by alleviating oxidation injury or by inhibiting oxidized-LDL-induced vascular smooth muscle cell (VSMC) proliferation, which may be promising mechanisms for treating atherosclerosis (Duan, W., et al., Yakugaku Zasshi (2005) 125(7):587-591). Corilagin's effects on coagulation, thrombosis, hypertension, and atherosclerosis have been reviewed (Duan, Weigang, Phytopharmacology and Therapeutic Values II pp. 163-172 (Studium Press LLC, Houston, Tex., 2007)). Significant toxicity of corilagin was not found in pilot toxicological studies. Overall, for P. amarus, in dosages commonly used (3-6 g of plant powder twice daily) no adverse reactions have been reported. See, Selected medicinal plants of India. A monograph of identity, safety and clinical usage (pp. 235-237; Bombay: Chemexcil. Basic Chemicals, Pharmaceuticals and Cosmetics Export Promotion Council, 1992).

Effects of corilagin and geraniin on TNF-α inhibitory activity have been compared to that of epigallocatechin gallate (O. Sachiko, et al., Biological & Pharmaceutical Bulletin (2001), 24(10):1145-1148). The IC₅₀ values of TNF-α release inhibition were: 43 μM for geraniin and 76 μM for corilagin, whereas that for (−)-epigallocatechin gallate (EGCG) was 26 μM. Treatment with geraniin prior to application of okadaic acid, a tumor promoter on mouse skin initiated with 7,12-dimethylbenz(a)anthracene, reduced the percentage of tumor-bearing mice from 80.0% to 40.0% and the average numbers of tumor per mouse from 3.8 to 1.1 in week 20 and, thus, geraniin has slightly weaker inhibitory activity than EGCG.

Geraniin, depicted in the compound of formula (2), is another tannoid member of the tannin family derived from galloyl glucose. Geraniin is named systematically as beta-D-Glucopyranose, cyclic 2-7:4-5 -(3,6-dihydro-2,9,10,11,11-pentahydroxy-3-oxo-2,6-methano-2H-1-benzoxocin-5,7-dicarboxylate) cyclic 3,6-(4,4′,5,5′,6,6′-hexahydroxy(1,1′-biphenyl)-2,2′-dicarboxylate) 1-(3,4,5-trihydroxybenzoate).

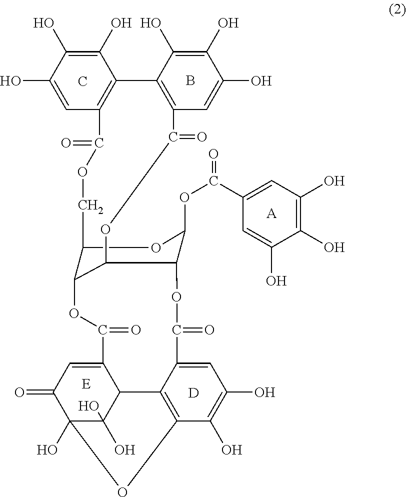

Geraniin, isolated from Phyllanthus sellowianus, was reported to be about six- to seven-fold more potent at the ID₅₀ level (micromol/kg) as an analgesic than aspirin and acetaminophen, respectively, although less efficacious when compared with the standard drugs (O. G. Miguel et al., Planta Med. (1996) 62(2):146-9). Antimicrobial activity of geraniin against Escherichia coli, Staphylococcus aureus and Candida albicans proved comparable activity to those of ampicillin, gentamycin and mycostatin (A. A. Gohara, et al., Z. Naturforsch. (2003) 58c, 670-674). Antiviral activity of gerannin against herpes simplex virus has been demonstrated (Yang, C-M., et al., J. Ethnopharmacology (2007) 110(3): 555-558). Antioxidant, anti-semi-carbazide-sensitive amine oxidase and anti-hypertensive properties of geraniin have been observed in spontaneously hypertensive rats. Anti-cancer properties of geraniin have also been reported by several research groups.

Use of P. amarus in the treatment of hepatitis, as discussed above, is based on early findings. Geraniin has been found to inhibit hepatitis B surface antigen (HBsAg) and hepatitis B e-antigen (HBeAg) secretion by more than 85.8% and 63.7%, respectively, at the non-cytotoxic concentration of 200 μg/ml. The inhibitions of HBsAg and HBeAg secretion by geraniin were higher than the inhibition by the positive control Lamivudine, 33.5% and 32.2% respectively, at the same concentration. Since HBeAg is involved in immune tolerance during HBV infection, the newly identified anti-HBV compound geraniin may be a candidate agent to overcome the immune tolerance in HBV infected individuals (J. Li, et al., Biological & Pharmaceutical Bulletin (2008) 31(4):743-747). Geraniin can also stimulate cellular activity, differentiation of and collagen synthesis in human skin keratinocytes and dermal fibroblasts, imparting wound healing properties.

Nirurin, depicted in the compound of formula (3), is a flavonoid constituent compound in P. amarus. Nirurin is chemically named as 5,6,7,4′-tetrahydroxy-8-(3-methylbut-2-enyl)flavanone-5 -O-rutinoside.

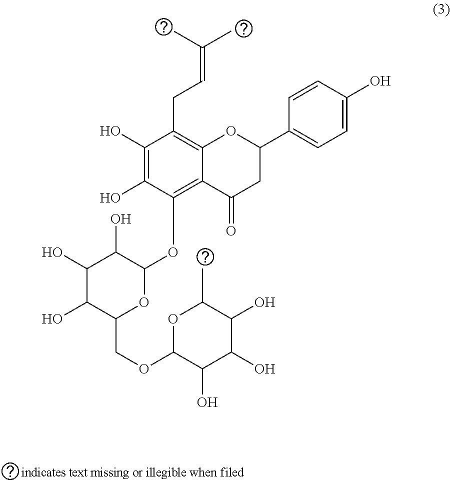 [SMILES: CC(C)=CCc1c(O)c(O)c(OC2OC(COC3OC(C)C(O)C(O)C3O)C(O)C(O)C2O)c2c1OC(c1ccc(O)cc1)CC2=O]

As evidenced by the extensive and significant pharmacological activity of several bioactive constituents and/or components of P. amarus, there is a need for these bioactives to be isolated to the maximum possible extent in an extract of this plant. In one embodiment, the present invention contemplates a P. amarus extract including an enriched hydrolyzable tannin blend. The enriched hydrolyzable tannin blend can include bioactive hydrolyzable tannins selected from corilagin, geraniin, nirurin and other low molecular weight hydrolyzable tannoids.

Several samples of P. amarus extract available in the market from different suppliers were obtained and quantitative analysis of the bioactives was performed using high pressure liquid chromatography (HPLC), the results of which are presented in Table 1.

HPLC Analytical Method

Each sample is derived from a commercially available standardized aqueous extract of Phyllanthus amarus whole plant (except roots) for nutraceutical and cosmetic use. The active constituents include a combination of corilagin, geraniin, nirurin, and other Low Molecular weight Hydrolysable Tannoids (LMwHT).

Sample Preparation. 50 mg of Phyllanthus amarus powdered extract (aqueous extract) is taken and dispersed in 10 ml of double distilled water. The dispersion is sonicated for 10 minutes and then centrifuged at 8500 rpm for 10 minutes. The resulting supernatant at a concentration of 5 mg/ml is injected (20 μl) for a typical HPLC run cycle.

HPLC Conditions.

Column: reversed phase C18 LiChroCART, 250 mm 1. X 4 mm i.d., 5 μm particle d. (E. Merck, Germany).

Column temp.: ambient.

Eluant: aqueous phase [A]: 0.1% phosphoric acid; organic phase [B] acetonitrile (ACN).

Flow rate: 1 ml/min.

Run Time: 37 min. Gradient: B 8-20% (20 min.), 20-22% (4 min.), 22-50% (10 min.), and re-equilibration 22-8% (3 min.).

UV detection at 270 nm; Waters HPLC Model 515 with PDA detector (Waters™ 2996, Photodiode Array Detector), evaluation with Empower.

HPLC Evaluation Method. The method was developed with external standards and evaluation of area of peaks using respective calibration equation.

A. Preparation of linear regression equation of corilagin. Reference standard of corilagin (available from TRC; North York, Canada) was dissolved in double distilled water to prepare four different concentrations (0.125, 0.25, 0.50 and 1.0 mg/ml), required for preparation of calibration curve. The amount of corilagin in Phyllanthus amarus extracts was determined using the regression equation of the calibration curve obtained as follows: Y=17020637.148x+1373249.087 with a correlation coefficient of 0.999. Y is the peak area and X is the concentration in mg/ml.

B. Preparation of linear regression equation of nirurin. Nirurin was isolated from Phyllanthus amarus by multiple column chromatography and was used as external standard. Nirurin, was dissolved in methanol to prepare four different concentrations (20, 10, 5 and 2 μg/ml), required for preparation of calibration curve. Calibration curve was plotted between area and different concentration. The linear regression equation of the calibration curve was obtained as follows: Y=36180x+24821 with a correlation coefficient of 0.999. Y is the peak area and X is the concentration in μg/ml.

Calculation Formulae

1. Corilagin: The area of the peak appearing at t_{R} 15.81 minutes is considered as Corilagin and the amount calculated using the above mentioned calibration equation of Corilagin (Y=17020637.148x+1373249.087) and the formula as follows. Corilagin present in the extract (% w/w)=[Amount of Corilagin obtained using calibration equation (mg)/Amount of extract injected (mg)]×100.

2. Geraniin: The area of the peak appearing at t_{R} 17.27 minutes is considered as Geraniin and the amount calculated using the above mentioned calibration equation of Corilagin (Y=17020637.148x+1373249.087) and the formula as follows. Geraniin present in the extract (% w/w)=[Amount of Geraniin obtained using calibration equation (mg)/Amount of extract injected (mg)]×100.

3. Other LMwHTs: The sum of the area of peaks appearing between 2.1 to 7.3 minutes, and 20.40 to 28.58 minutes are added and the amount of other LMwHTs calculated using the above linear regression equation of Corilagin (Y=17020637.148x+1373249.087) and the formula as follows. Other LMwHTs present in the extract (% w/w)=[Combined Amount of other LMwHTs obtained using calibration equation (mg) / Amount of extract injected (mg)]×100.

4. Nirurin: The area of the peak appearing at t_{R} 14.14 minutes is considered as Nirurin and the amount of Nirurin in sample is calculated using the above mentioned calibration equation of Nirurin (Y=36180x+24821) and the formula as follows. Nirurin present in the extract (% w/w)=[Amount of Nirurin obtained using calibration equation (μg)/Amount of extract injected (μg)]×100.

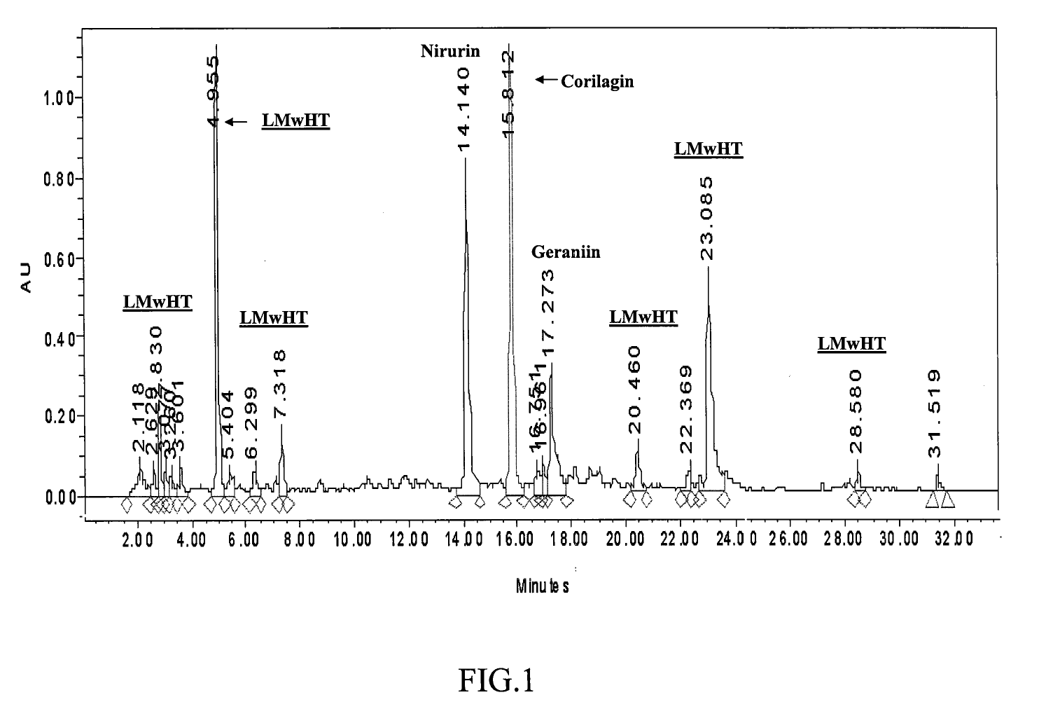

An exemplary chromatogram obtained using the analytical method as described herein is shown in FIG. 1.

Comparative HPLC

TABLE 1

| | Market | Market | Market | Market |
|---|---|---|---|---|
| | Sample 1 | Sample 2 | Sample 3 | Sample 4 |
| Bioactive | % w/w | % w/w | % w/w | % w/w |
| Corilagin | 2.21 | 0.11 | 0.58 | 1.01 |
| Geraniin | 0.31 | 0.00 | 0.00 | 0.00 |
| Nirurin | 4.58 | 0.87 | 0.43 | Traces |
| Other LMWtHTs | 10.15 | 1.18 | 1.59 | 7.97 |

As shown from the results in Table 1 above, P. amarus extracts currently available in the market contain very low amounts of most of the bioactives. Thus, there is a need for P. amarus extracts in which the bioactives are isolated, enriched, and/or preserved to a greater extent, if not the maximum extent possible. There is also a need to develop improved extraction conditions for enriching the extract with several different bioactives, including but not limited to corilagin geraniin, nirurin and other low molecular weight hydrolyzable tannoids. It is also imperative, from an environmental point of view, to have a method of extraction which is essentially completely aqueous.

Herbal extracts can be made by grinding one or more herbs, or at least one herb and an excipient and/or carrier, into a fine powder and suspending the powder into a solution of alcohol and water. It is understood by those skilled in the art that appropriate parts or portions of the herbal plants (optionally dried) may be used in the grinding process. The solution is regularly agitated or pulverized (e.g., by ultrasonication) over time and then pressed through a filtering medium to extract the bio-active ingredients.

In an embodiment, a process making a P. amarus extract containing a hydrolyzable tannin blend is provided. The invention further relates to a method for extracting Phyllanthus amarus to obtain an enriched hydrolyzable tannin powder.

The extraction process includes the steps of: providing over-ground portions of P. amarus; pulverizing or grinding the P. amarus to a powder; extracting the P. amarus powder with an extraction solvent or solvent mixture, optionally, with heating, to provide a P. amarus enriched extract; and concentrating or drying the P. amarus enriched extract to provide a hydrolyzable tannin enriched P. amarus powder. Aqueous solvent, or a solvent mixture, is preferred. A particularly preferred solvent is water. Useful extraction temperatures can range from about 50° C. to about 90° C. Particularly useful extraction temperatures can range from about 60° C. to about 80° C. Useful extraction times in conjunction with the useful temperatures can range from about 2 hours to about 10 hours. A particularly useful extraction time range at about 60±5° C. is from about 2 hours to about 4 hours. A particularly useful extraction time range at about 80±5° C. is from about 4 hours to about 8 hours. Another suitable extraction time range at about 80±5° C. is from about 2 hours to about 10 hours.

The extraction process can also include drying the extracted sample. Suitable drying methods include spray drying, freeze drying, lyophilization, vacuum drying, drying under heating, and concentration under vacuum. Once isolated or obtained the hydrolyzable tannin enriched P. amarus extract powder may be processed by any suitable means, including grinding, milling, sieving, sizing, and the like. The obtained hydrolyzable tannin enriched P. amarus extract powder may be prepared in any suitable particle size or particle size range.

In an exemplary extraction process, time and temperature are varied at atmospheric pressure (i.e., approx. 1 atm). It is contemplated that pressure can be varied in the extraction process, for example, by use of a pressure reactor apparatus. Suitable pressures used in the extraction process can range up to about 10 atm. Another suitable pressure for use in the process is 5 atm.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, water, fruit and/or vegetable juices, and combinations thereof

Solid nutritional compositions for oral administration may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearates, stearic acid, silicone fluid, talc, waxes, oils, colloidal silica, and the like. The usefulness of such excipients is well known in the art.

In a preferred embodiment, the nutritional composition may be in the form of a liquid. In accordance with this embodiment, a method of making a liquid composition is provided.

Liquid nutritional compositions for oral administration in connection with a method for preventing and/or treating colds and/or flu can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, or coloring and/or flavoring agents. Various liquid and powder nutritional compositions can be prepared by conventional methods. Various ready-to-drink formulations (RTD's) are contemplated.

The methods described above may be further understood in connection with the following Examples. The results of an extraction process may depend upon the solvent used, temperature of extraction, pressure at which extraction is performed, and duration of the extraction process. In several embodiments of this invention, these factors can be optimized to isolate and/or enrich and/or preserve the bioactives of P. amarus. P. amarus as used in the following examples was obtained from Ramakrishna Mission Ashrama, Narendrapur, Kolkata, West Bengal, India. As used herein, over-ground portions of P. amarus may include leaves and/or stems.

### EXAMPLE 1

The effect of temperature and duration of extraction were first optimized using water as an extraction solvent. In a typical experiment, two shade dried over ground portions of P. amarus (100 g) were pulverized and the resulting powder was extracted with distilled water (700 ml) for 12 hours at two different temperatures (60±5° C. and 80±5° C.) using a thermostatic water bath. Aliquots of the samples were withdrawn at different time intervals during extraction, spray dried and analyzed for bioactives (Corilagin, Geraniin, Nirurin and other LMwHTs) by HPLC (as discussed above). The results are incorporated in Tables 2 and 3, respectively.

Effect of duration of aqueous extraction at 60±5° C. on the bioactive contents of P. amarus

TABLE 2

| Bioactives | 0 Hr* | 2 Hr | 4 Hr | 6 Hr | 8 Hr | 10 Hr | 12 Hr |
|---|---|---|---|---|---|---|---|
| Corilagin | 0.00 | 3.22 | 3.20 | 3.94 | 2.26 | 2.21 | 1.79 |
| (% w/w) | | | | | | | |
| Geraniin | 4.825 | 16.51 | 11.23 | 8.26 | 2.42 | 2.38 | 1.83 |
| (% w/w) | | | | | | | |
| Nirurin | 0.00 | 2.84 | 2.75 | 4.16 | 3.73 | 4.74 | 4.81 |
| (% w/w) | | | | | | | |
| Other | 12.624 | 23.44 | 20.26 | 10.62 | 9.15 | 10.52 | 9.98 |
| LMwHTs | | | | | | | |
| (% w/w) | | | | | | | |

*indicates instant extraction

Effect of duration of aqueous extraction at 80±5° C. on the bioactive contents of P. amarus

TABLE 3

| Bioactives | 0 Hr* | 2 Hr | 4 Hr | 6 Hr | 8 Hr | 10 Hr | 12 Hr |
|---|---|---|---|---|---|---|---|
| Corilagin | 0.00 | 7.37 | 11.83 | 12.52 | 11.69 | 11.80 | 10.86 |
| (% w/w) | | | | | | | |
| Geraniin | 4.83 | 3.62 | 2.43 | 1.93 | 0.90 | 0.94 | 0.00 |
| (% w/w) | | | | | | | |
| Nirurin | 0.00 | 5.69 | 8.42 | 9.71 | 9.09 | 9.82 | 8.12 |
| (% w/w) | | | | | | | |
| Other | 12.62 | 17.97 | 20.48 | 19.24 | 14.65 | 9.06 | 6.65 |
| LMwHTs | | | | | | | |
| (% w/w) | | | | | | | |

*indicates instant extraction

As shown above, corilagin and nirurin were extracted more effectively and more efficiently in distilled water at 80±5° C. than at 60±5° C. at all the time points tested. The optimum duration of extraction for corilagin, nirurin and (to some degree) LMwHTs ranged from about 4-8 hours, and even up to about 10 hours, reaching maximum concentrations of corilagin and nirurin after 6 hours at 80±5° C. (Table 3). Geraniin was more efficiently extracted in distilled water at 60±5° C. than 80±5° C. The optimum duration of extraction for geraniin and LMwHTs ranged from 1-4 hours, reaching maximum concentrations at 2 hours at 60±5° C. (Table 2). These findings suggest that for a composition having maximum corilagin, nirurin, LMwHTs, and with appreciable geraniin contents, extraction at 80±5° C. for 6 hours would be optimal. Similarly, for a composition having maximum geraniin and LMwHTs contents, the optimum extraction conditions would be extraction at 60±5° C. for 2 hours. These extraction procedures yielded an enriched hydrolyzable tannin blend.

Since extraction at 80±5° C. for 6 hr. yielded maximum bioactive content in the extract, this condition was also used to determine the effect of different solvents on the bioactive content of P. amarus extract.

### EXAMPLE 2

The effect of different solvent extractions on the bioactives content was determined by using the optimized conditions i.e., 80±5° C. and up to 6 hrs of extraction. In a typical experiment, shade dried over-ground portions of the P. amarus plant were pulverized and extracted separately with one of the following solvents: distilled water; 50:50 (v/v) methanol:water; 30:70 (v/v) methanol:water; 50:50 (v/v) ethanol:water; or 30:70 (v/v) ethanol:water. All extractions were carried out with an herb powder:solvent ratio of 1:7 (i.e., 100 g P. amarus herb powder extracted with 700 ml solvent). All extractions were conducted for 6 hours at 80±5° C. using a thermostatic water bath. Aliquots of the samples were withdrawn at different time intervals during extraction, spray dried and analyzed for bioactives (Corilagin, Geraniin, Nirurin and other LMwHTs) by HPLC, as above. The results are incorporated in Table. 4.

Results in Table 4 indicate that aqueous extraction of dried over ground portions of P. amarus at 80±5° C. for 6 hours yielded better Corilagin (12.52%), Nirurin (9.71%) and LMwHTs (19.24%) contents than those of other solvent extractions. Ethanol:water (30:70 v/v) extraction at 80±5° C. for 6 hours yielded 10.57%, 9.14% and 16.12% of Corilagin, Nirurin and LMwHTs respectively. Methanol:water (30:70 v/v) extraction at 80±5° C. for 6 hours yielded 7.11%, 3.66% and 9.63% of Corilagin, Nirurin and LMwHT respectively. Geraniin was more efficiently extracted at 80±5° C. for 0 hr (instant extraction) in methanol:water (50:50 v/v), methanol:water (30:70 v/v) and ethanol:water (50:50 v/v) than water.

TABLE 4

| | Content of the Bioactives, % w/w | | | | | | | | | | | |
|---|---|---|---|---|---|---|---|---|---|---|---|---|
| | | | | | Methanol:Water | | | | Methanol:Water (30:70 v/v) | | | |
| Name of | Aqueous Extraction | | | | (50:50 v/v) extraction | | | | extraction | | | |
| Bioactive | 0 hr | 2 hr | 4 hr | 6 hr | 0 hr | 2 hr | 4 hr | 6 hr | 0 hr | 2 hr | 4 hr | 6 hr |
| Corilagin | 0.00 | 7.37 | 11.83 | 12.52 | 0.00 | 4.11 | 5.36 | 5.48 | 0.83 | 6.15 | 7.05 | 7.11 |
| Geraniin | 4.83 | 3.62 | 2.43 | 1.93 | 20.41 | 8.84 | 2.76 | 1.67 | 13.06 | 2.45 | 0.54 | 0.00 |
| Nirurin | 0.00 | 5.69 | 8.42 | 9.71 | 0.00 | 0.002 | 2.39 | 2.18 | 0.63 | 2.84 | 6.14 | 3.66 |
| Other | 12.62 | 17.9 | 20.48 | 19.24 | 18.91 | 14.89 | 11.33 | 11.95 | 13.93 | 13.98 | 10.40 | 9.63 |
| LMwHTs | | | | | | | | | | | | |

| | Content of the Bioactives, % w/w | | | | | | | |
|---|---|---|---|---|---|---|---|---|
| | Ethanol:Water | | | | Ethanol:Water (30:70 v/v) | | | |
| Name of | (50:50 v/v) extraction | | | | extraction | | | |
| Bioactive | 0 hr | 2 hr | 4 hr | 6 hr | 0 hr | 2 hr | 4 hr | 6 hr |
| Corilagin | 0.00 | 2.99 | 4.66 | 5.05 | 0.00 | 6.59 | 7.56 | 10.57 |
| Geraniin | 12.66 | 4.30 | 0.67 | 0.65 | 1.17 | 2.64 | 1.77 | 2.12 |
| Nirurin | 0.00 | 2.30 | 3.92 | 3.82 | 0.00 | 4.94 | 7.06 | 9.14 |
| Other | 13.04 | 12.24 | 12.22 | 17.65 | 9.09 | 9.53 | 10.67 | 16.12 |
| LMwHTs | | | | | | | | |

### EXAMPLE 2A

The effect of different atmospheric pressures (5 and 10 atmospheric pressure) on the extraction of bioactive components was determined using optimized conditions i.e., 80° C. and up to 6 hrs of extraction using water as an extraction solvent. In a typical experiment, two shade dried over ground portions of P. amarus (50 g) were pulverized and the resulting powder was extracted with distilled water (350 ml) at 80° C., with continuous stirring (400 RPM) for 6 hours, at two different pressures (5 and 10 atmospheric pressure) using a pressure reactor. Aliquots of the samples were withdrawn at different time intervals during extraction, spray dried and analyzed for bioactives (Corilagin, Geraniin, Nirurin and other LMwHTs) by HPLC (as discussed above). The results are incorporated in Tables 5 and 6, respectively.

Effect of 5 Atmospheric pressure (5 atm) aqueous extraction at 80° C. on the bioactive contents of P. amarus.

TABLE 5

| Bioactives | 0 Hr* | 2 Hr | 4 Hr | 6 Hr |
|---|---|---|---|---|
| Corilagin | 0.00 | 6.84 | 11.12 | 10.00 |
| (% w/w) | | | | |
| Geraniin | 0.42 | 5.49 | 6.32 | 3.29 |
| (% w/w) | | | | |
| Nirurin | 4.10 | 6.29 | 8.09 | 8.72 |
| (% w/w) | | | | |
| Other | 9.57 | 10.94 | 19.44 | 13.36 |
| LMwHTs | | | | |
| (% w/w) | | | | |

*indicates instant extraction

Effect of 10 Atmospheric pressure (10 atm) aqueous extraction at 80° C. on the bioactive contents of P. amarus.

TABLE 6

| Bioactives | 0 Hr* | 2 Hr | 4 Hr | 6 Hr |
|---|---|---|---|---|
| Corilagin | 0.00 | 11.8 | 16.7 | 12.95 |
| (% w/w) | | | | |
| Geraniin | 0.32 | 3.94 | 6.26 | 3.76 |
| (% w/w) | | | | |
| Nirurin | 4.44 | 10.79 | 10.60 | 9.07 |
| (% w/w) | | | | |
| Other | 9.37 | 13.17 | 19.26 | 13.92 |
| LMwHTs | | | | |
| (% w/w) | | | | |

*indicates instant extraction

As shown above, corilagin and nirurin were extracted more effectively and more efficiently in distilled water at 10 atm than at 5 atm and at all the time points tested. These findings suggest that water extraction at higher pressure (10 atmospheric pressure) may result in better corilagin, nirurin, LMwHTs, and with appreciable geraniin contents, than extraction at normal atmospheric pressure (1 atm).

In another embodiment, it is expected that a combination of the above extractive procedures may be carried out, without limitation, in a variety of multiple or sequential extraction steps, in order to obtain other optimized levels of the desired bioactive components. In particular, it is expected that by using the techniques applied above, optimized levels, or higher levels, of any one or all of the bioactive components, can be achieved.

### EXAMPLE 3

The effect of different extract drying procedures (Freeze drying, Spray drying Vacuum drying, and heating) on the content of bioactives was determined. Dry whole plants of Phyllanthus amarus were first pulverized and blended in a mini-blender. The resulting mixture was passed through a 22 mesh sieve to get uniform particle size of powder. 50 g powder was then extracted with 350 ml water (1:7 Solid-to-solvent ratio) at 80° C. for 6 hrs in a pressure reactor. The mixture was continuously stirred at a speed of 400 rpm. After completion of extraction, total sample was withdrawn, centrifuged for 5 minutes at 8000 rpm and filtered through filter paper. Total filtrate (water extract) was divided in four parts and dried by different method as follows:

20 ml extract was lyophilized;

20 ml extract was placed on a petri dish and kept on steam bath for 1 hr;

20 ml extract was concentrated on rotary evaporator under reduced pressure and kept overnight in vacuum dryer; and

Remaining extract was spray dried.

The dried extracts were analyzed for bioactives (Corilagin, Geraniin, Nirurin and other LMwHTs) by HPLC (as discussed above). The results are incorporated in Table 7.

Effect of different drying conditions on the bioactives (Corilagin, Nirurin, Geraniin, Gallic acid, Ellagic acid and LMWHTs) content of P. amarus extract.

TABLE 7

| | | | | Other |
|---|---|---|---|---|
| | Corilagin | Geraniin | Nirurin | LMWHTs |
| Name of Sample | % (w/w) | % (w/w) | % (w/w) | % (w/w) |
| PA/Lyophilized extract | 11.44 | 4.86 | 7.96 | 9.67 |
| PA/Spray dried extract | 11.15 | 4.12 | 6.58 | 8.44 |
| PA/Vacuum dried | 8.32 | 3.91 | 7.49 | 8.41 |
| extract | | | | |
| PA/Extract dried on | 7.04 | 1.59 | 5.69 | 7.51 |
| steam bath | | | | |

The above results indicate that freeze drying (lyophilizing) and spray drying yielded better Corilagin, Geraniin, Nirurin contents in the dried extract than vacuum drying or drying over steam heat.

In view of the examples, it is expected that use of a hydrolyzable tannin enriched P. amarus extract made in accordance with the principles of the invention, in a pharmaceutical or nutraceutical composition, would possess improved properties for the treatment or prevention of diseases, in particular, liver and kidney diseases.

### EXAMPLE 4

Comparative Hepatoprotective Activity of P. Amarus Extracts on CCl₄-Induced Liver Injury in Mice

Five different P. amarus-based extract powders were tested, including (a) the enriched hydrolyzable tannin blend of Example 1 (Table 3), and (b) Market Samples 1-4 (Table 1, as discussed above). Each sample was a free-flowing powder.

Experimental animals. Swiss Albino mice of both sex weighing approximately 32±4 g, 10-15 weeks old were obtained from National Research Institute of Ayurveda for Drug Development (Govt of India), Kolkata, and were housed in polypropylene cages at 22±3° C., relative air humidity of 45 to 55%, with 12.00 hr light & dark cycle (lighting on from 6:00 AM to 6:00 PM). Mice were provided a standard pellet chow (carbohydrate 65.5%, protein 17.6%, fat 6.6%) and distilled water ad libitum. The mice were acclimatized for one week in the laboratory conditions, before being used in the experiment. All experiments were conducted between 10.00 hr and 14.00 hr. Principles of laboratory animals care (NIH publication no. 85-23, revised 1985) were followed.

Drug Preparation. Test samples were suspended in 0.3% CMC solutions of distilled water and were administered orally for 10 days by using an intubation canula and volume of dose was 0.1 ml/10 g body weight.

CCl₄-Induced Hepatotoxicity. Hepatotoxicity was induced by administration of CCl₄ in liquid paraffin (1:2) at the dose of 1.0 ml/kg intraperitoneally once in every 72 h for 10 days.

Drug Protocol. The mice were divided into 7 groups (n=6). Details of the drug treatments and dose regimen are listed below in Table 8. Mice in Group A served as a vehicle control, which received 0.3% Carboxymethylcellulose solution (CMC) at the dose of 0.1 ml/10 g. Group B served as CC1₄ control and was not treated with any drug. Groups C, D, E, F, and G were administered with PA/enriched hydrolyzable tannin blend, and PA/Market Samples 1 to 4, respectively, at the dose level of 250 mg/kg body weight, p.o, for 10 days. CCl₄ in liquid paraffin (1:2) at the dose of 1.0 ml/kg intraperitoneally once in every 72 h for 10 days was administered to mice from Group B-G.

Treatment groups are listed in Table 8, as follows.

TABLE 8

| Groups | Treatment | Doses |
|---|---|---|
| Group A | Normal | Vehicle Control 0.3% CMC (0.1 ml/10 g) |
| | animals | [p.o] |
| Group B | CCl₄ | CCl₄ (30%) + in liquid paraffin (1:2) |
| | | (1 ml/kg) [i.p] |
| Group C | CCl₄ + PA/ | CCl₄ (30%) in liquid paraffin (1:2) (1 ml/kg) |
| | enriched | [i.p.] + PA/enriched hydrolyzable tannin |
| | hydrolyzable | blend (250 mg/kg) [p.o.] |
| | tannin blend | |
| Group D | CCl₄ + PA/ | CCl₄ (30%) in liquid paraffin (1:2) (1 ml/kg) |
| | Market | [i.p.] + PA/Market Sample 1 (250 mg/kg) |
| | Sample 1 | [p.o.] |
| Group E | CCl₄ + PA/ | CCl₄ (30%) in liquid paraffin (1:2) (1 ml/kg) |
| | Market | [i.p.] + PA/Market Sample 2 (250 mg/kg) |
| | Sample 2 | [p.o.] |
| Group F | CCl₄ + PA/ | CCl₄ (30%) in liquid paraffin (1:2) (1 ml/kg) |
| | Market | [i.p.] + PA/Market Sample 3 (250 mg/kg) |
| | Sample 3 | [p.o.] |
| Group G | CCl₄ + PA/ | CCl₄ (30%) in liquid paraffin (1:2) (1 ml/kg) |
| | Market | [i.p.] + PA Market Sample 4 (250 mg/kg) |
| | Sample 4 | [p.o.] |

Procedure. After 24 hours of the last dose, blood was collected from retro-orbital plexus under ether anesthesia. The blood samples were allowed to clot and the serum was separated by centrifugation at 2500 rpm at 37° C. and used for the assay of biochemical marker enzymes (SGOT, SGPT and ALP), bilirubin and total protein by using commercially available kits (Span Diagnostic Ltd., Surat, India). The assay results that were found are listed in Table 9. These data include the effects of various Phyllanthus amarus extracts on some serum biochemical parameters of the CCl₄-intoxicated mice that were tested.

TABLE 9

| | | | | | Total | | |
|---|---|---|---|---|---|---|---|
| | | SGPT | SGOT | ALP | protein | Bilirubin | MDA |
| Groups | Treatment | (IU/L) | (IU/L) | (IU/L) | (mg/dl) | (mg/dl) | (nmol/ml) |
| Group A | Normal animals | 11.29 ± | 6.96 ± | 11.26 ± | 5.25 ± | 0.65 ± | 3.75 ± |
| | | 0.86 | 0.78 | 0.70 | 0.08 | .00 | .11 |
| Group B | CCl₄ | 33.05 ± | 27.65 ± | 33.80 ± | 2.64 ± | 1.38 ± | 7.26 ± |
| | | 1.55^{¥¥¥} | 2.35^{¥¥¥} | 0.51 | 0.06^{¥¥¥} | 0.06^{¥¥¥} | .11^{¥¥¥} |
| Group C | CCl₄ + | 11.46 ± | 8.00 ± | 12.19 ± | 5.20 ± | 0.66 ± | 3.79 ± |
| | PA/enriched | 1.28***^{aabbccdd} | 1.12***^{d} | 0.47***^{aaabbbcccddd} | 0.13*** | 0.01***^{abbccd} | .17***^{aaabbbcccddd} |
| | hydrolyzable | | | | | | |
| | tannin blend | | | | | | |
| Group D | CCl₄ + PA/ | 17.99 ± | 10.15 ± | 17.67 ± | 4.93 ± | 0.83 ± | 5.15 ± |
| | Market Sample 1 | 0.92*** | 0.54*** | 0.83*** | 0.06*** | 0.04***^{a} | 0.11*** |
| Group E | CCl₄ + PA/ | 17.05 ± | 13.45 ± | 16.98 ± | 4.85 ± | 0.84 ± | 5.00 ± |
| | Market Sample 2 | 1.59*** | 1.43*** | 1.10*** | 0.06*** | .01*** | .10*** |
| Group F | CCl₄ + PA/ | 18.2 ± | 11.80 ± | 17.16 ± | 4.91 ± | 0.87 ± | 4.98 ± |
| | Market Sample 3 | 1.70*** | 1.06*** | 0.31*** | 0.06*** | .04*** | .05*** |
| Group G | CCl₄ + PA/ | 17.81 ± | 10.25 ± | 18.87 ± | 4.99 ± | 0.86 ± | 5.02 ± |
| | Market Sample 4 | 0.87*** | 0.73*** | 0.87*** | 0.10*** | 0.04*** | .05*** |

p values are Mean ± SEM; n = 6 in each group
p values were obtained by ANOVA followed by post hoc comparison between groups by Newman-Keuls comparison test.
^{¥¥¥}p < 0.001; in comparison to vehicle treated mice
***p < 0.001; in comparison to CCl₄ treated mice
^{aaa}p < 0.001;
^{aa}p < 0.01;
^{a}p < 0.05 in comparison to PA/Market Sample 1 treated mice
^{bbb}p < 0.001;
^{bb}< 0.01;
^{b}p < 0.05 in comparison to PA/Market Sample 2 treated mice
^{ccc}p < 0.001;
^{cc}p < 0.01;
^{c}p < 0.05 in comparison to PA/Market Sample 3 treated mice
^{ddd}p < 0.001;
^{dd}p < 0.01;
^{d}p < 0.05 in comparison to PA/Market Sample 4 treated mice

As shown in Table 9, CCl₄ administration (in liquid paraffin (1:2) at the dose of 1.0 ml/kg intraperitoneally once in every 72 h for 10 days), produced liver damage in mice (Group B) as indicated by the increase in the levels of hepatic marker enzymes (SGOT, SGPT and ALP) in comparison to vehicle control (Group A). Administration of Phyllanthus amarus extracts of Market Samples 1-4 (Groups D-G) significantly attenuated the CC1₄-induced hepatotoxicity as evidenced by the significant decrease in the SGOT, SGPT, ALP and Bilirubin levels and significant increase in the total protein in comparison to CCl₄ treated group (Group B). However, notably, the PA/enriched hydrolyzable tannin blend (Group C) reversed, and essentially restored, the enzyme, bilirubin, and total protein levels to those of the control (Group A). The extract of PA/enriched hydrolyzable tannin blend (Group C) showed a dramatic and unexpected improvement in lowering of hepatic enzyme levels over the Market Samples 1-4, as follows. For Market Samples 1-4, taking the normal control group measurements as a baseline, the reduction of SGPT after CCl₄ treatment ranged from 68-75%, while in comparison for the PA/enriched hydrolyzable tannin blend (Group C) a 99% reduction of SGPT after CCl₄ treatment was measured. For Market Samples 1-4, taking the normal control group measurements as a baseline, the reduction of SGOT after CCl₄ treatment ranged from 68-84%, while in comparison for the PA/enriched hydrolyzable tannin blend (Group C) a 95% reduction of SGOT after CCl₄ treatment was measured. For Market Samples 1-4, taking the normal control group measurements as a baseline, the reduction of ALP after CCl₄ treatment ranged from 66-75%, while in comparison for the PA/enriched hydrolyzable tannin blend (Group C) a 96% reduction of ALP after CCl₄ treatment was measured. Thus, the improvement in reduction of elevated enzyme levels provided by the PA/enriched hydrolyzable tannin blend (Group C) was found to be in the range of 25-31% for SGPT, 11-27% for SGOT, and 21-30% for ALP.

In summary, the PA/enriched hydrolyzable tannin blend (Group C) showed much better activity than those extracts of Market Samples 1-4 (Groups D-G) as indicated by statistically significant differences in comparison to other marketed sample treated groups. Stated in another way, treatment provided by the PA/enriched hydrolyzable tannin blend (Group C) solved the problem of elevated liver enzymes that the less potent preparations of other known products could not solve.

Serum ALP and bilirubin levels are also related to the status and function of hepatic cells. Increased levels of ALP and bilirubin are correlated with increased lipid peroxidation and inflammation. As shown above in Table 9, Phyllanthus amarus extracts were found to reduce both serum ALP (as discussed above) and bilirubin in the CCl₄ treated groups. The free radicals produced in vivo from CCl₄ attack the cell membrane and leads to membrane damage, alteration in the structure and function of cellular membrane. Thus increased levels of lipid peroxides are indicators of liver damage due to high oxidative stress in CCl₄ intoxicated mice. Administration of Phyllanthus amarus extracts of Market Samples 1-4 (Groups D-G) significantly attenuated the CCl₄-induced hepatotoxicity as evidenced by the significant decrease in Bilirubin levels. As mentioned above, the PA/enriched hydrolyzable tannin blend (Group C) reversed, and essentially restored, bilirubin levels to those of the control (Group A). For Market Samples 1-4, taking the normal control group measurements as a baseline, the reduction of Bilirubin after CCl₄ treatment ranged from 70-75%, while in comparison for the PA/enriched hydrolyzable tannin blend (Group C) a 99% reduction of Bilirubin after CCl₄ treatment was measured. Thus, the improvement in reduction of bilirubin provided by the PA/enriched hydrolyzable tannin blend (Group C) was found to be in the range of 24-29%. In summary, the PA/enriched hydrolyzable tannin blend (Group C) showed much better activity than those extracts of Market Samples 1-4 (Groups D-G) as indicated by significantly lowering the CCl₄ induced lipid peroxidation in comparison to other marketed sample treated groups. The results show the potent anti-inflammatory and antioxidant nature of the PA/enriched hydrolyzable tannin blend.

In conclusion, the results of the study demonstrate that all the Phyllanthus amarus extracts showed significant hepatoprotective activity against carbon tetrachloride induced liver damage in mice. However, the extract of the PA/enriched hydrolyzable tannin blend (Group C) showed much better activity than those extracts of Market Samples 1-4 (Groups D-G), demonstrating the superior properties of the PA/enriched hydrolyzable tannin blend composition.

It is further expected that a hydrolyzable tannin enriched P. amarus extract made in accordance with the principles of the invention would be effective as a nutritional supplement.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A Phyllanthus amarus extract composition comprising a hydrolyzable tannin blend including about 3-20% by weight of corilagin based on the total weight of the extract.

2. A Phyllanthus amarus extract composition comprising a hydrolyzable tannin blend including about 1-25% by weight of geraniin based on the total weight of the extract.

3. A Phyllanthus amarus extract composition comprising a hydrolyzable tannin blend including about 3-20% by weight corilagin based on the total weight of the extract, about 3.5-10% by weight nirurin based on the total weight of the extract, about 1-3% by weight geraniin based on the total weight of the extract, and about 9-20% by weight low molecular weight hydrolyzable tannoids based on the total weight of the extract.

4. A pharmaceutical or nutraceutical composition comprising the extract of claim 1 and a pharmaceutically acceptable carrier.

5. A pharmaceutical or nutraceutical composition comprising the extract of claim 2 and a pharmaceutically acceptable carrier.

6. A method of making a Phyllanthus amarus extract composition comprising a hydrolyzable tannin blend, including the steps of:
(a) providing portions of a P. amarus plant;
(b) grinding the P. amarus plant portions to provide a powder;
(c) extracting the P. amarus powder with water to provide a P. amarus aqueous extract; and
(d) drying the P. amarus aqueous extract to provide a P. amarus extract as a powder, said powder contains a hydrolyzable tannin blend including about 7-13% by weight corilagin based on the total weight of the extract powder, about 3.5-10% by weight nirurin based on the total weight of the extract powder, about 1-2% by weight geraniin based on the total weight of the extract powder, and about 9-20% by weight low molecular weight hydrolyzable tannoids based on the total weight of the extract powder.

7. The method of claim 6, wherein the extracting step is carried out at about 80° C. for a time of about 4 hours to about 8 hours.

8. The method of claim 7, wherein the extracting step is carried out from about 5 atm to about 10 atm.

9. The method of claim 6, wherein the drying step is carried out by a method selected from the group consisting of freeze drying, vacuum drying, lyophilization, spray drying, and drying over a heat source.

10. A method of treating or preventing liver damage in an individual, comprising administering to the individual in need of such treatment a therapeutically effective amount of a composition according to claim 1, wherein the levels of at least one liver enzyme is decreased.

11. The method of claim 10, wherein the liver enzyme is selected from one or more of serum glutamic-oxaloacetic transaminase (SGOT), serum glutamate pyruvate transaminase (SGPT) alanine transaminase (ALT), aspartate transaminase (AST), and alkaline phosphatase (ALP).

12. A method of treating or preventing liver damage in an individual, comprising administering to the individual in need of such treatment a therapeutically effective amount of a composition according to claim 2, wherein the levels of at least one liver enzyme is decreased.

13. The method of claim 12, wherein the liver enzyme is selected from one or more of serum glutamic-oxaloacetic transaminase (SGOT), serum glutamate pyruvate transaminase (SGPT) alanine transaminase (ALT), aspartate transaminase (AST), and alkaline phosphatase (ALP).
